(19) United States
(12) Patent Application Publication
(10) Pub. No.: US 2026/0144807 A1
(43) Pub. Date: May 28, 2026

(54) METHOD OF ADMINISTRATION OF A PARENTERAL FORMULATION COMPRISING A PSYCHEDELIC AGENT

(71) Applicant: Cybin UK LTD, London (GB)

(72) Inventors: Peter RANDS, London (GB); Ellen JAMES, London (GB); Tiffanie BENWAY, London (GB); Zelah JOEL, London (GB); Carol ROUTLEDGE, London (GB); David ERRITZOE, London (GB); Christopher TIMMERMANN SLATER, London (GB); Marie LAYZELL, London (GB)

(73) Assignees: CYBIN UK LTD, London (GB); Cybin UK LTD, London (GB)

(21) Appl. No.: 19/119,888

(22) PCT Filed: Oct. 13, 2023
(86) PCT No.: PCT/EP2023/078480

§ 371 Date: Apr. 10, 2025

Related U.S. Application Data
(60) Provisional application No. 63/379,358, filed on Oct. 13, 2022.
(60) Provisional application No. 63/481,046, filed on Jan. 23, 2023.
(60) Provisional application No. 63/488,628, filed on Mar. 6, 2023.

(51) Int. Cl. A61K 31/675; A61K 31/4045; A61P 25/22; A61P 25/24

(52) U.S. Cl. CPC A61K 31/675; A61K 31/4045; A61P 25/22; A61P 25/24

(57) ABSTRACT

The invention relates to a dosage regimen, method for treating, delivery device or parenteral formulation for use in the treatment of a psychiatric or neurological disorder in a patient. In particular, the invention relates to the administration of a psychedelic agent.

### FIELD OF THE INVENTION

The invention relates to a dosage regimen, method for treating, delivery device or parenteral formulation for use in the treatment of a psychiatric or neurological disorder in a patient. In particular, the invention relates to the administration of a psychedelic agent.

### BACKGROUND OF THE INVENTION

Classical psychedelics have shown preclinical and clinical promise in treating psychiatric disorders (Carhart-Harris and Goodwin, Neuropsychopharmacology 42, 2105-2113 (2017)). In particular, psilocybin has demonstrated significant improvement in a range of depression and anxiety rating scales in randomised double-blind studies (Griffiths et al. Journal of Psychopharmacology, 30 (12), 1181-1197 (2016)). Efficacy of psilocybin has been shown in depression (R. L. Carhart-Harris et al., Psychopharmacology, 2018, 235, 399-408), end of life anxiety (R. R. Griffiths et al., J. Psychopharmacol., 2016, 30, 12, 1181-1197) and addiction (M. W. Johnson, A. Garcia-Romeu and R. R. Griffiths, Am. J. Drug Alcohol Abuse, 2017, 43, 1, 55-60), and is currently being investigated for several other mental health disorders that are rooted in psychologically destructive patterns of thought processing (Anorexia Nervosa: NCT #NCT04052568).

N,N-dimethyltryptamine (DMT) is also proposed to hold therapeutic value as a short-acting psychedelic. A review of research into the biosynthesis and metabolism of DMT in the brain and peripheral tissues, methods and results for DMT detection in body fluids and the brain is provided by S. A. Barker in Front. Neurosci., 12, 536, 1-17 (2018). Barker et al. suggest that ‘Further characterization of DMT cellular distribution, receptors and general biochemistry may lead to new targets for more effective pharmaceutical substances and interventions’. D. Nutt et al. (Cell. 2020 Apr. 2; 181 (1): 24-28. doi: 10.1016/j.cell.2020.03.020) suggest a possible therapeutic role for DMT: ‘It is theoretically possible, however, that a short trip, such as with i.v. DMT, might “shake-up” and “reset” abnormal patterns of brain activity and so could have some therapeutic benefit.’ Both Barker et al. and Nutt et al. conclude that further research is required into the role and function of DMT.

Sanches et al and Palhano-Fontes et al report that in studies of patients with recurrent major depressive disorder (MDD) or treatment-resistant depression (TRD), a single oral dose of ayahuasca, a natural psychedelic-containing plant brew was associated with improvements in depressive symptoms (Sanches R F, de Lima Osorio F, dos Santos R G, et al. Antidepressant effects of a single dose of ayahuasca in patients with recurrent depression: A SPECT study. J Clin Psychopharmacol 2016; 36 (1): 77-81 and Palhano-Fontes F, Barreto D, Onias H, et al. Rapid antidepressant effects of the psychedelic ayahuasca in treatment-resistant depression: a randomized placebo-controlled trial. Psychol Med 2019; 49 (4): 655-663). N,N-dimethyltryptamine (DMT) is the principal psychedelic compound contained in ayahuasca.

Good et al. discuss the results of a phase 1 study to investigate the pharmacokinetics of N,N-dimethyltryptamine fumarate in healthy subjects (Meghan Good, Tiffanie Benway, Zelah Joel, et al., Authorea. May 12, 2022; DOI: 10.22541/au. 165237523.39763980/v1). No patients with a psychiatric or neurological disorder were included in the study. This report has not been peer-reviewed.

D'Souza et al report an ‘exploratory study of the dose-related safety, tolerability, and efficacy of dimethyltryptamine (DMT) in healthy volunteers and major depressive disorder’ (D. C. D'Souza, S. A. Syed, L. T. Flynn, H. Safi-Aghdam, N. V. Cozzi and M. Ranganathan, Neuropsychopharmacology (2022) 47:1854-1862). Seven patients with major depressive disorder received two doses of DMT hemifumarate (0.1 mg/kg, followed by 0.3 mg/kg) at least 48 hours apart. The participants were administered DMT by intravenous push over 30-60 seconds, i.e., a bolus injection.

WO2022195489 discusses methods for the use of psychedelics, in which an initial bolus injection is administered, followed by the administration of a lower maintenance dose. WO2022031566 discusses the intravenous administration of DMT.

Timmermann et al. discuss the results of a clinical trial to investigate the effects of intravenous DMT in healthy participants (Translational Psychiatry (2023) 13:172, published online on 23 May 2023).

Cybin Inc. have completed the Phase 1 part of a Phase 1/2a clinical trial evaluating a deuterated psilocybin compound (CYB003) in healthy participants with and without major depressive disorder (ClinicalTrials.gov Identifier: NCT05385783). In February 2023, Cybin Inc. announced that following a single oral dose of CYB003, psychedelic effects were seen within ≈15 minutes and the average duration of peak effects lasted≈2 hours, data based on an interim analysis of CYB003 in healthy volunteers (https://cybin.com/cyb003/).

In May 2023, Cybin Inc. announced that the company is evaluating the intravenous administration of a deuterated dimethyltryptamine compound (CYB004) in healthy volunteers. Part C of the Phase 1 CYB004-E trial is a crossover study design which will evaluate IV bolus+infusion regimens of CYB004 in up to two cohorts.

With the growing interest in the potential use of psychedelic agents in psychiatry, such as psilocybin and DMT, there is an increasing need for methods of administering these agents in a safe and tolerable manner.

### SUMMARY OF THE INVENTION

As a first aspect, the invention provides a dosage regimen for administering a psychedelic agent to a patient for the treatment of a psychiatric or neurological disorder, comprising parenterally administering a dose of the psychedelic agent for a dosing period of from about 5 to about 15 minutes, wherein the psychedelic agent is selected from N,N-dimethyltryptamine, 5-methoxy-N,N-dimethyltryptamine, 4-acetoxy-N,N-dimethyltryptamine, psilocybin, psilocin, deuterated analogues thereof, and pharmaceutically acceptable salts thereof, and wherein the parenteral administration provides a slow onset breakthrough psychedelic experience in the patient.

As a second aspect, the invention provides a method for treating a psychiatric or neurological disorder in a patient, comprising parenterally administering to said patient a dose of a psychedelic agent for a dosing period of from about 5 to 15 minutes, wherein the psychedelic agent is selected from N,N-dimethyltryptamine, 5-methoxy-N,N-dimethyltryptamine, 4-acetoxy-N,N-dimethyltryptamine, psilocybin, psilocin, deuterated analogues thereof, and pharmaceutically acceptable salts thereof, and wherein the parenteral administration provides a slow onset breakthrough psychedelic experience in the patient.

As a third aspect, the invention provides a delivery device for use in the treatment of a psychiatric or neurological disorder in a patient, wherein the delivery device is configured to deliver a parenterally administered dose of a psychedelic agent for a dosing period of from about 5 to about 15 minutes, wherein the psychedelic agent is selected from N,N-dimethyltryptamine, 5-methoxy-N,N-dimethyltryptamine, 4-acetoxy-N,N-dimethyltryptamine, psilocybin, psilocin, deuterated analogues thereof, and pharmaceutically acceptable salts thereof, and wherein the parenteral administration provides a slow onset breakthrough psychedelic experience in the patient.

As a fourth aspect, the invention provides a parenteral formulation for use in the treatment of a psychiatric or neurological disorder in a patient, comprising parenteral administration of a dose of a psychedelic agent for a dosing period of from about 5 to about 15 minutes, wherein the psychedelic agent is selected from N,N-dimethyltryptamine, 5-methoxy-N,N-dimethyltryptamine, 4-acetoxy-N,N-dimethyltryptamine, psilocybin, psilocin, deuterated analogues thereof, and pharmaceutically acceptable salts thereof, wherein the parenteral administration provides a slow onset breakthrough psychedelic experience in the patient.

As a fifth aspect, the invention provides a parenteral formulation comprising a psychedelic agent for use in the treatment of a psychiatric or neurological disorder in a patient, comprising parenteral administration of a dose of the psychedelic agent for a dosing period of from about 5 to about 15 minutes, wherein the psychedelic agent is selected from N,N-dimethyltryptamine, 5-methoxy-N,N-dimethyltryptamine, 4-acetoxy-N,N-dimethyltryptamine, psilocybin, psilocin, deuterated analogues thereof, and pharmaceutically acceptable salts thereof, wherein the parenteral administration provides a slow onset breakthrough psychedelic experience in the patient.

### FIGURES

FIG. 1 shows the percentage of subjects with ≥50% reduction in Montgomery-Asberg Depression Rating Scale (MADRS) score compared with baseline assessment, at week 1 and week 2 after administration of DMT fumarate or placebo.

FIG. 2 shows the percentage of subjects with a MADRS score≤10 at week 1 and week 2 after administration of DMT fumarate or placebo.

### DEFINITIONS

Throughout this specification, one or more aspects of the invention may be combined with one or more features described in the specification to define distinct embodiments of the invention.

In the detailed description, reference is made to a number of terms, which are to be understood to have the meanings provided below, unless a context expressly indicates to the contrary.

The nomenclature used herein for the psychedelic agents for use in the invention is as commonly used in the field. The compounds described herein, may also be referred to by nomenclature in accordance with the rules of the International Union of Pure and Applied Chemistry (IUPAC) for chemical compounds, specifically the “IUPAC Compendium of Chemical Terminology (Gold Book)” (see A. D. Jenkins et al., Pure & Appl. Chem., 1996, 68, 2287-2311). For the avoidance of doubt, if a rule of the IUPAC organisation is contrary to a definition provided herein, the definition herein is to prevail.
N,N-dimethyltryptamine is also known by the IUPAC name 2-(1H-indol-3-yl)-N,N-dimethylethanamine.
5-Methoxy-N,N-dimethyltryptamine is also known by the IUPAC name 2-(5-methoxy-1H-indol-3-yl)-N,N-dimethylethanamine.
4-Acetoxy-N,N-dimethyltryptamine is also known by the IUPAC name [3-[2-(dimethylamino)ethyl]-1H-indol-4-yl]acetate.
Psilocybin is also known by the IUPAC name [3-[2-(dimethylamino)ethyl]-1H-indol-4-yl]dihydrogen phosphate.
Psilocin is also known by the IUPAC name 3-[2-(dimethylamino)ethyl]-1H-indol-4-ol.

As used herein the term ‘deuterated analogues’ of the psychedelic agent means that one or more hydrogen atom(s) in the structure of the psychedelic agent is replaced with a deuterium atom, wherein a deuterium atom is a hydrogen atom with an additional neutron. Substitution with deuterium may be at one or more of the α and β positions, on the methyl groups, and on the indole ring, and in certain compounds at a substituent to the indole ring, e.g., the methoxy group of 5-methoxy-N,N-dimethyltryptamine. In some embodiments, deuteration may be at the methyl groups, and at the α, and optionally the β positions.

Where compounds described herein are indicated as being or described as deuterated analogues, the compound concerned is enriched with deuterium by an amount that is dependent on the percentage of deuterium available in the reagents from which the compounds are derived. For example, the do-dimethylamino portions of compounds of formula I, wherein —NR²R³ is —N(CD₃)₂, may be derived from dimethyl-d₇-amine, or dimethyl-de-amine (commonly available as HCl salts), which are available from chemical vendors in purities of deuterium that range from 98% to 99%. The purity of deuterium in the resultant d₆-dimethylamino substituents is consequently between 98% and 99%. This means, as the skilled person will understand, that not all compounds of formula I (for example) will comprise d₆-dimethylamino substituents-some may comprise d₀-dsdimethylamino, but the average purity of deuterium is about 98% to 99%.

In some embodiments, the psychedelic agent is substituted at position 5 with methoxy, or at position 4 with acetoxy or hydroxy, or with monohydrogen phosphate. The term “acetoxy” (often abbreviated to OAc) defines a univalent group derived from acetic acid by removal of a hydrogen atom from the OH moiety. The term “methoxy” (often abbreviated to OMe) defines a univalent group derived from methanol by removal of a hydrogen atom from the OH moiety. The term “hydroxy” (often abbreviated to OH) defines a univalent group derived from water by removal of a hydrogen atom from the H₂O moiety. The term monohydrogen phosphate defines a divalent group of formula HPO₄, derived from phosphoric acid by removal of a proton from two of the three OH moieties, and thus denotes a substituent of formula —OP(O)(OH)O⁻.

When the dimethyltryptamine compound is substituted at position 4 with hydroxy, the psychedelic agent is referred to herein as psilocin. When the dimethyltryptamine compound is substituted at position 4 with monohydrogen phosphate, this is to reflect that psilocybin (also known as [3-(2-Dimethylaminoethyl)-1H-indol-4-yl] dihydrogen phosphate) in water generally has monohydrogen phosphate at the 4-position, this generally being understood to be the predominant form owing to the pKa values of the two terminal phosphate oxygen atoms being estimated as 1.3 and 6.5. It is further understood that the monohydrogen phosphate-containing form of psilocybin exists as a zwitterion (i.e., an internal salt) in which the nitrogen atom of the dimethylamino moiety is protonated. This form is thus, and psilocybin is to be regarded as, a salt of a dimethyltryptamine compound substituted at position 4 with monohydrogen phosphate.

For the avoidance of doubt, positions 4 and 5, and α and β, of the psychedelic agent refer to the positions labelled in the structure below (substitution not shown).

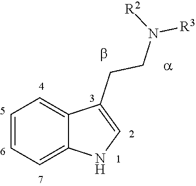

References herein to the dose or total dose of a psychedelic agent refer to the dose or total dose as the free base equivalent of that agent.

As used herein the term ‘parenteral administration’ or ‘parenterally administering’ means the administration of a dose of a psychedelic agent via any route other than oral, in one or more portions over the duration of the dosing period. The parenteral administration is essentially continuous for a dosing period of from 5 to 15 minutes. For the avoidance of doubt, the ‘parenteral administration’ excludes an initial bolus administration of a psychedelic agent, that is a single dose of an agent administered at a rapid rate, typically over 30 to 60 seconds, which single dose provides a fast onset breakthrough psychedelic experience.

Fast onset means a breakthrough psychedelic experience which peaks within two minutes following the start of administration of the psychedelic agent. As used herein, the term ‘slow onset breakthrough psychedelic experience’ refers to a breakthrough psychedelic experience which peaks two minutes or longer following the start of the administration of the psychedelic agent,

References herein to a singular of a noun encompass the plural of the noun, and vice-versa, unless the context implies otherwise.

Throughout this specification the word ‘comprise’, or variations such as ‘comprises’ or ‘comprising’, will be understood to imply the inclusion of a stated element, integer or step, or group of elements, integers or steps, but not the exclusion of any other element, integer or step, or group of elements, integers or steps. The term ‘comprising’ includes within its ambit the term ‘consisting’ or ‘consisting essentially of’.

The term ‘consisting’ or variants thereof is to be understood to imply the inclusion of a stated element, integer or step, or group of elements, integers or steps, and the exclusion of any other element, integer or step or group of elements, integers or steps.

The term ‘consisting essentially of’ or variants thereof is to be understood to imply the inclusion of a stated element, integer or step, or group of elements, integers or steps, and that further components may be present, but only those not materially affecting the essential characteristics of the embodiment of the invention.

The term ‘about’ herein, when qualifying a number or value, is used to refer to values that lie within ±5% of the value specified. For the avoidance of doubt, where a number or value is specified herein in the absence of the term ‘about’, the number or value should be understood according to standard numeric rounding conventions according to the number of decimal places. For example, a whole number, such as 6, is understood to encompass values≥5.5 and <6.5. Likewise, a number specified to one decimal place, such as 5.3, is understood to encompass values≥5.25 and <5.35.

Where a range of values is provided, the range includes the end point values. For example, the range 20 to 28, or from 20 to 28, would include the values 20, 21, 22, 23, 24, 25, 26, 27 and 28; the range 5 to 15, or from 5 to 15, would include the values 5, 6, 7, 8, 9, 10, 11, 12, 13, 14 and 15.

As used herein, the term ‘patient’ preferably refers to a mammal. Typically, the mammal is a human, but may also refer to a domestic mammal. The term does not encompass laboratory mammals.

As used herein, the term ‘in combination with psychotherapy’ refers to the treatment of a psychiatric disorder by psychological means, which are enhanced by the dosing regimen, method for treating, delivery device, or parenteral formulation for use of the invention.

The term “treatment” defines the therapeutic treatment of a patient, in order to reduce or halt the rate of progression of a disorder, or to ameliorate or cure the disorder. Prophylactic treatment of a patient having a diagnosed psychiatric or neurological disorder is also included. Such prophylactic treatment, also referred to as secondary prevention, aims to reduce the impact of the disorder and/or to hinder development of the disorder through treatment in accordance with the invention.

As used herein ‘neurological disorder’ refers to a disorder which may be associated with a dysfunction in the brain or nervous system, and which may result in physical and/or psychological symptoms. As used herein, the term ‘psychiatric disorder’ is characterised by a clinically significant disturbance in an individual's cognition, emotional regulation, or behaviour, and that is associated with present distress (e.g., a painful symptom) or disability (i.e., impairment in one or more important areas of functioning) or with a significantly increased risk of suffering death, pain, disability, or an important loss of freedom.

Diagnostic criteria for psychiatric or neurological disorders referred to herein are provided in the Diagnostic and Statistical Manual of Mental Disorders, Fifth Edition, (DSM-5).

As used herein the term ‘psychedelic assisted psychotherapy’, is defined as any psychotherapeutic practice that is provided alongside a dose of a psychedelic therapeutic formulation, including for example any dose as defined in the present invention. The term ‘psychedelic assisted psychotherapy’ includes support therapy providing preparation, psychological support and therapeutic integration to patients around and during administration of the psychedelic agent.

As used herein the term ‘obsessive-compulsive disorder’ (OCD) is defined by the presence of either obsessions or compulsions, but commonly both. The symptoms can cause significant functional impairment and/or distress. An obsession is defined as an unwanted intrusive thought, image or urge that repeatedly enters the person's mind. Compulsions are repetitive behaviours or mental acts that the person feels driven to perform. Typically, OCD manifests as one or more obsessions, which drive adoption of a compulsion. For example, an obsession with germs may drive a compulsion to clean or an obsession with food may drive a compulsion to overeat, eat too little or throw up after eating (i.e., an obsession with food may manifest itself as an eating disorder). A compulsion can either be overt and observable by others, such as checking that a door is locked, or a covert mental act that cannot be observed, such as repeating a certain phrase in one's mind.

As used herein, the term ‘eating disorder’ is defined by severe and persistent disturbance in eating behaviours and associated distressing thoughts and emotions. The term ‘eating disorder’ includes anorexia nervosa and bulimia nervosa, binge eating disorder, avoidant restrictive food intake disorder, other specified feeding and eating disorder, pica and rumination disorder.

Eating disorders often co-occur with anxiety disorders and obsessive compulsive disorder. Neziroglu and Sandler differentiate between OCD and an eating disorder: ‘Whereas patients with eating disorders are primarily driven by concerns of physical appearance, and consequently alter their eating patterns in order to lose weight accordingly. OCD patients may be restricting their eating for reasons very different than body image concerns’ (https://iocdf.org/expert-opinions/expert-opinion-eating-disorders-and-ocd/).

The term “eating disorder” includes anorexia nervosa, bulimia and binge eating disorder (BED). The symptoms of anorexia nervosa include eating too little and/or exercising too much in order to keep weight as low as possible. The symptoms of bulimia include eating a lot of food in a very short amount of time (i.e., binging) and then being deliberately sick, using laxatives, eating too little and/or exercising too much to prevent weight gain. The symptoms of BED include regularly eating large portions of food until uncomfortably full, and consequently feeling upset or guilty.

As used herein the term ‘depressive disorder’ includes major depressive disorder, persistent depressive disorder, bipolar disorder, bipolar depression, and depression in terminally ill patients.

As used herein the term ‘major depressive disorder’ (MDD, also referred to as major depression or clinical depression) is defined as the presence of five or more of the following symptoms over a period of two-weeks or more (also referred to herein as a ‘major depressive episode’), most of the day, nearly every day:
depressed mood, such as feeling sad, empty or tearful (in children and teens, depressed mood can appear as constant irritability);
significantly reduced interest or feeling no pleasure in all or most activities;
significant weight loss when not dieting, weight gain, or decrease or increase in appetite (in children, failure to gain weight as expected);
insomnia or increased desire to sleep;
either restlessness or slowed behaviour that can be observed by others;
fatigue or loss of energy;
feelings of worthlessness, or excessive or inappropriate guilt;
trouble making decisions, or trouble thinking or concentrating;
recurrent thoughts of death or suicide, or a suicide attempt.

At least one of the symptoms must be either a depressed mood or a loss of interest or pleasure.

Persistent depressive disorder, also known as dysthymia, is defined as a patient exhibiting the following two features:

A. has depressed mood for most of the time almost every day for at least two years. Children and adolescents may have irritable mood, and the time frame is at least one year.

B. While depressed, a person experiences at least two of the following symptoms:
Either overeating or lack of appetite.
Sleeping too much or having difficulty sleeping.
Fatigue, lack of energy.
Poor self-esteem.
Difficulty with concentration or decision-making.

As used herein the term ‘treatment resistant major depressive disorder’ describes MDD that fails to achieve an adequate response to treatment with standard of care therapy.

As used herein, ‘bipolar disorder’, also known as manic-depressive illness, is a disorder that causes unusual shifts in mood, energy, activity levels, and the ability to carry out day-to-day tasks.

There are two defined sub-categories of bipolar disorder; all of them involve clear changes in mood, energy, and activity levels. These moods range from periods of extremely “up,” elated, and energised behaviour (known as manic episodes, and defined further below) to very sad, “down,” or hopeless periods (known as depressive episodes). Less severe manic periods are known as hypomanic episodes.

Bipolar I Disorder—defined by manic episodes that last at least 7 days, or by manic symptoms that are so severe that the person needs immediate hospital care. Usually, depressive episodes occur as well, typically lasting at least 2 weeks. Episodes of depression with mixed features (having depression and manic symptoms at the same time) are also possible.

Bipolar II Disorder-defined by a pattern of depressive episodes and hypomanic episodes, but not the full-blown manic episodes described above.

As used herein ‘bipolar depression’ is defined as an individual who is experiencing depressive symptoms with a previous or coexisting episode of manic symptoms, but does not fit the clinical criteria for bipolar disorder.

As used herein, the term ‘anxiety disorder’ includes generalised anxiety disorder, phobia, panic disorder, social anxiety disorder, and post-traumatic stress disorder.

‘Generalised anxiety disorder’ (GAD) as used herein means a chronic disorder characterised by long-lasting anxiety that is not focused on any one object or situation. Those suffering from GAD experience non-specific persistent fear and worry, and become overly concerned with everyday matters. GAD is characterised by chronic excessive worry accompanied by three or more of the following symptoms: restlessness, fatigue, concentration problems, irritability, muscle tension, and sleep disturbance.

‘Phobia’ is defined as a persistent fear of an object or situation the affected person will go to great lengths to avoid, typically disproportional to the actual danger posed. If the feared object or situation cannot be avoided entirely, the affected person will endure it with marked distress and significant interference in social or occupational activities.

A patient suffering from a ‘panic disorder’ is defined as one who experiences one or more brief attack(s) (also referred to as a panic attack) of intense terror and apprehension, often marked by trembling, shaking, confusion, dizziness, nausea, and/or difficulty breathing. A panic attack is defined as a fear or discomfort that abruptly arises and peaks in less than ten minutes.

‘Social anxiety disorder’ is defined as an intense fear and avoidance of negative public scrutiny, public embarrassment, humiliation, or social interaction. Social anxiety often manifests specific physical symptoms, including blushing, sweating, and difficulty speaking.

‘Post-traumatic stress disorder’ (PTSD) is an anxiety disorder that results from a traumatic experience. Post-traumatic stress can result from an extreme situation, such as combat, natural disaster, rape, hostage situations, child abuse, bullying, or even a serious accident. Common symptoms include hypervigilance, flashbacks, avoidant behaviours, anxiety, anger and depression.

As used herein, the term “post-partum depression” (PPD, also known as postnatal depression) is a form of depression experienced by either parent of a newborn baby. Symptoms typically develop within 4 weeks of delivery of the baby and often include extreme sadness, fatigue, anxiety, loss of interest or pleasure in hobbies and activities, irritability, and changes in sleeping or eating patterns.

As used herein, the term ‘substance abuse’ means a patterned use of a drug in which the user consumes the substance in amounts or with methods that are harmful to themselves or others.

As used herein, the term ‘gambling disorder’ means persistent and recurrent problematic gambling behaviour leading to clinically significant impairment or distress. The disorder has similarities with substance abuse.

As used herein, the term ‘an avolition disorder’ refers to a disorder that includes as a symptom the decrease in motivation to initiate and perform self-directed purposeful activities.

As used herein, the term ‘a psychedelic experience’ refers to a period in which the patient experiences one or more intense reactions or emotions, altered state of perception, visual or other sensory hallucinations, spiritual experience, ego dissolution and dissociation. Ego dissolution describes a state in which the boundary between an individual and the outside world dissolves. Dissociation describes a state in which an individual experiences a sensation that different parts of the brain are not connected, such as a disconnect between mind and body.

The Mystical Experience Questionnaire (MEQ) was designed to address experiences occasioned specifically by hallucinogens and allows researchers to understand the characteristically incomprehensible subjective experiences associated with psychedelics. The MEQ consists of 30 questions; participants are asked to answer each question according to one's feelings, thoughts, and experiences at the time of the session, each item being rated on a 0-5 scale (0-None/not at all to 5-Extreme, more than any other time in my life). The minimum score is 0 and the maximum score is 150 with higher scores indicating a greater degree of mystical experience. The MEQ total score is computed by taking the average response to all items. In some embodiments, a ‘psychedelic experience’ is an experience which has a percentage score of at least 40% on the Mystical Experience Questionnaire (MEQ), for example 40% or more, 45% or more, 50% or more, 55% or more, or 60% or more points on the MEQ.

‘Breakthrough psychedelic experience’ means an intense and immersive psychedelic experience in which almost all connection to the real world is lost. In some embodiments breakthrough psychedelic experience is an experience which has a percentage score of 45% or more, 50% or more, 55% or more, or 60% or more on the MEQ.

### DETAILED DESCRIPTION

The dosage regimen, method for treating, delivery device, or parenteral formulation for use of the invention provides a slower onset regimen (compared with administration of a bolus, for example an IV bolus), which may improve the therapeutic benefit in patients by slowing the onset of the peak experience, and may improve tolerability. In addition, the psychedelic experience will be slightly prolonged, which may improve the therapeutic potential. The dosage regimen, method for treating, delivery device, or parenteral formulation for use of the present invention provides an additional safety measure, by giving the physician the opportunity to stop the infusion, either at the patient's request or if required by clinical observations.

Accordingly, the first aspect of the invention provides as embodiment 1 a dosage regimen for administering a psychedelic agent to a patient for the treatment of a psychiatric or neurological disorder, comprising parenterally administering a dose of the psychedelic agent for a dosing period of from about 5 to about 15 minutes, wherein the psychedelic agent is selected from N,N-dimethyltryptamine, 5-methoxy-N,N-dimethyltryptamine, 4-acetoxy-N,N-dimethyltryptamine, psilocybin, psilocin, deuterated analogues thereof, and pharmaceutically acceptable salts thereof, and wherein the parenteral administration provides a slow onset breakthrough psychedelic experience in the patient.

The second aspect of the invention provides, as embodiment 2, a method for treating a psychiatric or neurological disorder in a patient, comprising parenterally administering to the patient a dose of a psychedelic agent for a dosing period of from about 5 to about 15 minutes, wherein the psychedelic agent is selected from N,N-dimethyltryptamine, 5-methoxy-N,N-dimethyltryptamine, 4-acetoxy-N,N-dimethyltryptamine, psilocybin, psilocin, deuterated analogues thereof, and pharmaceutically acceptable salts thereof, and wherein the parenteral administration provides a slow onset breakthrough psychedelic experience in the patient.

The third aspect of the invention provides, as embodiment 3, a delivery device for use in the treatment of a psychiatric or neurological disorder in a patient, wherein the delivery device is configured to deliver a parenterally administered dose of a psychedelic agent for a dosing period of from about 5 to about 15 minutes, wherein the psychedelic agent is selected from N,N-dimethyltryptamine, 5-methoxy-N,N-dimethyltryptamine, 4-acetoxy-N,N-dimethyltryptamine, psilocybin, psilocin, deuterated analogues thereof, and pharmaceutically acceptable salts thereof, and wherein the parenteral administration provides a slow onset breakthrough psychedelic experience in the patient.

The fourth aspect of the invention provides, as embodiment 4, a parenteral formulation for use in the treatment of a psychiatric or neurological disorder in a patient, comprising parenteral administration of a dose of a psychedelic agent for a dosing period of from about 5 to about 15 minutes, wherein the psychedelic agent is selected from N,N-dimethyltryptamine, 5-methoxy-N,N-dimethyltryptamine, 4-acetoxy-N,N-dimethyltryptamine, psilocybin, psilocin, deuterated analogues thereof, and pharmaceutically acceptable salts thereof, and wherein the parenteral administration provides a slow onset breakthrough psychedelic experience in the patient.

The fifth aspect of the invention provides, as embodiment 5, a parenteral formulation comprising a psychedelic agent for use in the treatment of a psychiatric or neurological disorder in a patient, comprising parenteral administration of a dose of the psychedelic agent for a dosing period of from about 5 to about 15 minutes, wherein the psychedelic agent is selected from N,N-dimethyltryptamine, 5-methoxy-N,N-dimethyltryptamine, 4-acetoxy-N,N-dimethyltryptamine, psilocybin, psilocin, deuterated analogues thereof, and pharmaceutically acceptable salts thereof, wherein the parenteral administration provides a slow onset breakthrough psychedelic experience in the patient.

As embodiment 6, the invention provides a dosage regimen, method for treating, delivery device or parenteral formulation for use, according to any preceding embodiment, wherein the parenteral administration is selected from the group consisting of intravenous, intramuscular, subcutaneous, intranasal, transmucosal, sublingual, rectal, and transdermal administration and inhalation.

As embodiment 7, the invention provides a dosage regimen, method for treating, delivery device or parenteral formulation for use, according to any preceding embodiment, wherein the administration is by intravenous infusion, preferably wherein the intravenous administration is conducted using a syringe pump.

As embodiment 8, the invention provides a dosage regimen, method for treating, delivery device or parenteral formulation for use, according to any preceding embodiment, wherein the parenteral administration is by a two-phase intravenous infusion.

As embodiment 9, the invention provides a dosage regimen, method for treating, delivery device or parenteral formulation for use, according to any one of embodiments 1 to 7, wherein the parenteral administration is by a single-phase intravenous infusion.

As embodiment 10, the invention provides a dosage regimen, method for treating, delivery device or parenteral formulation for use, according to any preceding embodiment, wherein the dosing period is from about 8 to about 12 minutes.

As embodiment 11, the invention provides a dosage regimen, method for treating, delivery device or parenteral formulation for use, according to any preceding embodiment, wherein the dosing period is from about 9 to about 11 minutes, or about 10 minutes.

As embodiment 12, the invention provides a dosage regimen, method for treating, delivery device or parenteral formulation for use, according to any preceding embodiment, comprising the parenteral administration of a total dose of the psychedelic agent selected from the group consisting of:
about 20 to about 70 mg of N,N-dimethyltryptamine, a deuterated analogue, or a pharmaceutically acceptable salt thereof;
about 15 to about 30 mg of N,N-dimethyltryptamine, a deuterated analogue, or a pharmaceutically acceptable salt thereof;
about 5 to about 15 mg of N,N-dimethyltryptamine, a deuterated analogue, or a pharmaceutically acceptable salt thereof;
about 10 to about 30 mg of 5-methoxy-N,N-dimethyltryptamine, a deuterated analogue, or a pharmaceutically acceptable salt thereof
about 1 to about 5 mg of psilocybin, a deuterated analogue, or a pharmaceutically acceptable salt thereof;
about 3 to about 15 mg of 4-acetoxy-N,N-dimethyltryptamine, a deuterated analogue, or a pharmaceutically acceptable salt thereof; and
about 1 to about 5 mg, or about 3 to about 25 mg of psilocin, a deuterated analogue, or a pharmaceutically acceptable salt thereof.

As embodiment 13, the invention provides a dosage regimen, method for treating, delivery device or parenteral formulation for use, according to any preceding embodiment, comprising the parenteral administration of a total dose of about 20 to about 29 mg of N,N-dimethyltryptamine, a deuterated analogue, or a pharmaceutically acceptable salt thereof.

As embodiment 14, the invention provides a dosage regimen, method for treating, delivery device or parenteral formulation for use, according to any preceding embodiment, comprising the parenteral administration of a total dose of about 20 to about 23 mg, or a total dose of about 26 to about 29 mg, of N,N-dimethyltryptamine, a deuterated analogue, or a pharmaceutically acceptable salt thereof.

As embodiment 15, the invention provides a dosage regimen, method for treating, delivery device or parenteral formulation for use, according to embodiment 8, wherein the administration is by a two-phase intravenous infusion, the psychedelic agent is N,N-dimethyltryptamine, a deuterated analogue, or a pharmaceutically acceptable salt thereof, and the total dose is about 20 to about 23 mg.

As embodiment 16, the invention provides a dosage regimen, method for treating, delivery device or parenteral formulation for use, according to embodiment 9, wherein the administration is by a single-phase intravenous infusion, the psychedelic agent is N,N-dimethyltryptamine, a deuterated analogue, or a pharmaceutically acceptable salt thereof, and the total dose is about 26 to about 29 mg.

As embodiment 17, the invention provides a dosage regimen, method for treating, delivery device or parenteral formulation for use, according to embodiment 9, wherein the administration is by a single-phase intravenous infusion, the psychedelic agent is N,N-dimethyltryptamine, a deuterated analogue, or a pharmaceutically acceptable salt thereof, and the total dose is about 7 to about 10 mg, or about 10 to about 20 mg.

As embodiment 18, the invention provides a dosage regimen, method for treating, delivery device or parenteral formulation for use, according to embodiment 9, wherein the administration is by intramuscular administration, the psychedelic agent is N,N-dimethyltryptamine, a deuterated analogue, or a pharmaceutically acceptable salt thereof, and the total dose is about 20 to about 70 mg.

As embodiment 19, the invention provides a dosage regimen, method for treating, delivery device or parenteral formulation for use, according to any one of embodiments 1 to 12, comprising the parenteral administration of a total dose of about 1.5 to about 3 mg of psilocybin, a deuterated analogue, or a pharmaceutically acceptable salt thereof; or about 1.5 to about 3 mg of psilocin, a deuterated analogue, or a pharmaceutically acceptable salt thereof.

As embodiment 20, the invention provides a dosage regimen, method for treating, delivery device or parenteral formulation for use, according to any preceding embodiment, wherein the psychedelic agent is a compound of formula I, or a pharmaceutically acceptable salt thereof:

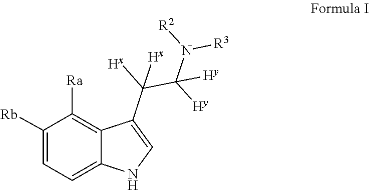

Ra is selected from H, D, —OH, —OAc and —PO₃OH, and Rb is H or D; or
Ra is H or D, and Rb is selected from H, D, and —Ome;
R² and R³ are each independently selected from C(H^{z})₃; and
each H^{x}, H^{y} and H^{z} is independently selected from protium and deuterium.

As embodiment 21, the invention provides a dosage regimen, method for treating, delivery device or parenteral formulation for use, according to embodiment 20, wherein R² and R³ are each independently selected from C(H)₃ and C(D)₃.

As embodiment 22, the invention provides a dosage regimen, method for treating, delivery device or parenteral formulation for use, according to embodiment 20, wherein R² and R³ are both C(H)₃, or R² and R³ are both C(D)₃.

As embodiment 23, the invention provides a dosage regimen, method for treating, delivery device or parenteral formulation for use, according to any one of embodiments 20 to 21, wherein each H^{x} is H, or each H^{x} is D, one H^{x} is H and one H^{x} is D.

As embodiment 24, the invention provides a dosage regimen, method for treating, delivery device or parenteral formulation for use, according to any one of embodiments 20 to 23, wherein each H^{y} is H, or each H^{y} is D, or one H^{y} is H and one H^{y} is D.

As embodiment 25, the invention provides a dosage regimen, method for treating, delivery device or parenteral formulation for use, according to any one of embodiments 20 to 24, wherein each H^{x}, H^{y} and H^{z} is deuterium.

As embodiment 26, the invention provides a dosage regimen, method for treating, delivery device or parenteral formulation for use, according to any preceding embodiment, wherein the psychedelic agent is selected from the group consisting of:

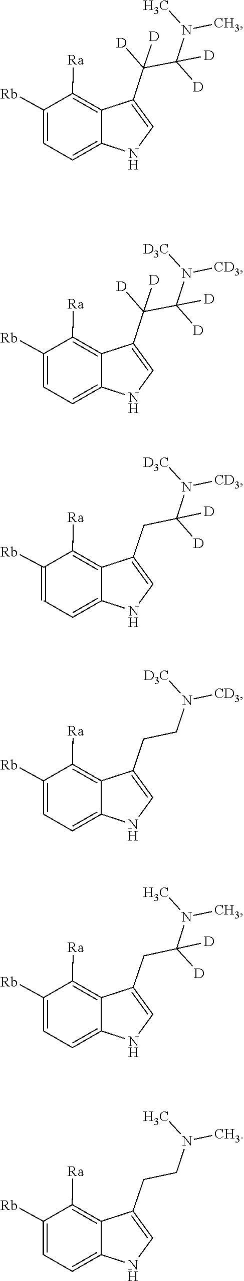

or a pharmaceutically acceptable salt thereof; wherein R^{a} and R^{b} are as defined in embodiment 20.

As embodiment 27, the invention provides a dosage regimen, method for treating, delivery device or parenteral formulation for use, according to any one of embodiments 20 to 26, wherein Ra is H.

As embodiment 28, the invention provides a dosage regimen, method for treating, delivery device or parenteral formulation for use, according to any one of embodiments 20 to 27, wherein Rb is H.

As embodiment 29, the invention provides a dosage regimen, method for treating, delivery device or parenteral formulation for use, according to any preceding embodiment, wherein the parenteral administration comprises intravenous infusion by one or two syringe pumps.

As embodiment 30, the invention provides a dosage regimen, method for treating, delivery device or parenteral formulation for use, according to any preceding embodiments, wherein the psychiatric or neurological disorder is selected from the group consisting of (i) an obsessive compulsive disorder, (ii) a depressive disorder, (iii) an anxiety disorder, (iv) substance abuse and gambling disorders and (v) an avolition disorder. Often, the disorder is selected from the group consisting of major depressive disorder, treatment resistant major depressive disorder, post-partum depression, an obsessive compulsive disorder and an eating disorder such as a compulsive eating disorder. Preferably the psychiatric or neurological disorder is selected from the group consisting of a depressive disorder and an anxiety disorder.

As embodiment 31, the invention provides a dosage regimen, method for treating, delivery device or parenteral formulation for use, according to any preceding embodiments, wherein the psychiatric or neurological disorder is a depressive disorder.

As embodiment 32, the invention provides a dosage regimen, method for treating, delivery device or parenteral formulation for use, according to any preceding embodiments, wherein the psychiatric or neurological disorder is major depressive disorder.

As embodiment 33, the invention provides a dosage regimen, method for treating, delivery device or parenteral formulation for use, according to any preceding embodiment, wherein, the duration of the psychedelic experience is 3 hours or less, or 2 hours or less, or 1 hour or less.

As embodiment 34, the invention provides a dosage regimen, method for treating, delivery device or parenteral formulation for use, according to any preceding embodiment, wherein, the duration of the psychedelic experience is:
From about 15 to about 30 minutes, or from about 30 to about 90 minutes when the psychedelic agent is N,N-dimethyltryptamine, a deuterated analogue, or a pharmaceutically acceptable salt thereof; or
From about 15 to about 45 minutes, or about 45 minutes to about 180 minutes when the psychedelic agent is selected from psilocybin, 4-acetoxy-N,N-dimethyltryptamine, 5-methoxy-N,N-dimethyltryptamine, psilocin, a deuterated analogue thereof, or a pharmaceutically acceptable salt thereof; or
From about 20 to about 30 minutes wherein the psychedelic agent is N,N-dimethyltryptamine or a pharmaceutically acceptable salt thereof; or
From about 60 to about 90 minutes wherein the psychedelic agent is a deuterated analogue of N,N-dimethyltryptamine, or a pharmaceutically acceptable salt thereof.

As embodiment 35, the invention provides a dosage regimen, method for treating, delivery device or parenteral formulation for use, according to embodiment 33 or 34, wherein the duration of experience is assessed by an attending clinician, psychiatrist, or therapist.

As embodiment 36, the invention provides a dosage regimen, method for treating, delivery device or parenteral formulation for use, according to any preceding embodiment, further comprising:
a. Preparation Stage, comprising preparing the patient for a psychedelic experience;
b. Administration Stage, comprising the dosage regimen or method of treatment according to any preceding embodiment; and
c. Integration Stage, comprising a psychiatrist or therapist led interview or discussion with the patient focussed on the psychedelic experience.

As embodiment 37, the invention provides a dosage regimen, method for treating, delivery device or parenteral formulation for use, according to any preceding embodiment wherein the treatment of a psychiatric or neurological disorder in a patient comprises psychedelic assisted psychotherapy.

The psychedelic agents for use in the dosage regimen, method for treating, delivery device, or parenteral formulation for use of the invention may be prepared according to the synthetic processes described in WO2021/089873, US20210395201, WO2022/117359, U.S. Pat. Nos. 11,242,318, 11,724,985, and US20220202775, the disclosures of which are hereby incorporated by reference in their entireties.

The dose of psychedelic agent for use in the first, second, third and fourth aspects of the present invention may preferably be in the form of a pharmaceutically acceptable salt wherein the salt comprises an acid and the freebase of a psychedelic agent selected from N,N-dimethyltryptamine, 5-methoxy-N,N-dimethyltryptamine, 4-acetoxy-N,N-dimethyltryptamine, psilocybin, psilocin, and deuterated analogues thereof. An example of a salt comprising an acid and dimethyltryptamine compound is N,N-dimethyltryptamine fumarate, which is the fumaric acid salt of N,N-dimethyltryptamine. P. H. Stahl and C. G. Wermuth provide an overview of pharmaceutical salts and the acids comprised therein in Handbook of Pharmaceutical Salts: Properties, Selection and Use, Weinheim/Zürich: Wiley-VCH/VHCA, 2002. The acids described in this review are suitable acids for inclusion within the salt of the formulation.

To be abundantly clear, when the phrase, “psychedelic agent is selected from N,N-dimethyltryptamine, 5-methoxy-N,N-dimethyltryptamine, 4-acetoxy-N,N-dimethyltryptamine, psilocybin, psilocin, deuterated analogues thereof, and pharmaceutically acceptable salts thereof” is used, this means that 1) any one of the listed compounds, 2) a pharmaceutically acceptable salt of any one of the listed compounds, 3) a deuterated analogue of any one of the listed compounds, and 4) a pharmaceutically acceptable salt of any one of the deuterated analogues of any one of the listed compounds is included. For instance, as a non-limiting example, the “psychedelic agent” would include N,N-dimethyltryptamine, a pharmaceutically acceptable salt of N,N-dimethyltryptamine, a deuterated analogue of N,N-dimethyltryptamine, or a pharmaceutically acceptable salt of a deuterated analogue of N,N-dimethyltryptamine. The same is true for the other listed compounds.

A preferred psychedelic agent for use in any embodiment of the invention is selected from:
N,N-dimethyltryptamine;
α-protio, α-deutero-N,N-dimethyltryptamine;
α,α-dideutero-N,N-dimethyltryptamine;
α,α,β,β-tetradeutero-N,N-dimethyltryptamine;
N,N-di(trideuteromethyl) tryptamine;
α-protio, α-deutero-N,N-di(trideuteromethyl) tryptamine;
α,α-dideutero-N,N-di(trideuteromethyl) tryptamine;
α,α,β,β-tetradeutero-N,N-di(trideuteromethyl) tryptamine;
5-methoxy-N,N-dimethyltryptamine;
5-methoxy-α-protio, α-deutero-N,N-dimethyltryptamine;
5-methoxy-α,α-dideutero-N,N-dimethyltryptamine;
5-methoxy-α,α,β,β-tetradeutero-N,N-dimethyltryptamine;
5-methoxy-N,N-di(trideuteromethyl) tryptamine;
5-methoxy-α-protio, α-deutero-N,N-di(trideuteromethyl) tryptamine;
5-methoxy-α,α-dideutero-N,N-di(trideuteromethyl) tryptamine;
5-methoxy-α,α,β,β-tetradeutero-N,N-di(trideuteromethyl) tryptamine;
psilocybin;
α-protio, α-deutero-psilocybin;
α,α-dideutero-psilocybin;
α,α,β,β-tetradeutero-psilocybin;
4-(dihydrogen phosphate)-N,N-di(trideuteromethyl) tryptamine;
4-(dihydrogen phosphate)-α-protio, α-deutero-N,N-di(trideuteromethyl) tryptamine;
4-(dihydrogen phosphate)-α,α-dideutero-N,N-di(trideuteromethyl) tryptamine;
4-(dihydrogen phosphate)-α,α,β,β-tetradeutero-N,N-di(trideuteromethyl) tryptamine;
psilocin;
α-protio,α-deutero-psilocin;
α,α-dideutero-psilocin;
α,α,β, β-tetradeutero-psiloin;
4-hydroxy-N,N-di(trideuteromethyl) tryptamine;
4-hydroxy-α-protio, α-deutero-N,N-di(trideuteromethyl) tryptamine;
4-hydroxy-α,α-dideutero-N,N-di(trideuteromethyl) tryptamine;
4-hydroxy-α,α,β,β-tetradeutero-N,N-di(trideuteromethyl) tryptamine; and pharmaceutically acceptable salts thereof.

The salt may comprise an acid selected from the group consisting of fumaric acid, tartaric acid, citric acid, acetic acid, lactic acid, gluconic acid, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, adipic acid, ascorbic acid, aspartic acid, benzenesulfonic acid, benzoic acid, camphoric acid, camphor-10-sulfonic acid, decanoic acid, hexanoic acid, octanoic acid, carbonic acid, cinnamic acid, cyclamic acid, dodecylsulfuric acid, ethane-1,2-disulfonic acid, ethanesulfonic acid, formic acid, galactaric acid, gentisic acid, glucoheptonic acid, glucuronic acid, glutamic acid, glutaric acid, glycerophosphoric acid, glycolic acid, hippuric acid, hydrobromic acid, hydrochloric acid, isobutyric acid, lactobionic acid, lauric acid, maleic acid, malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid (−L), salicylic acid, sebacic acid, stearic acid, succinic acid, sulfuric acid, thiocyanic acid, toluenesulfonic acid and undecylenic acid.

Preferably, the pharmaceutically acceptable salt is selected from fumarate, tartrate, citrate and hydrochloride.

More preferably, the pharmaceutically acceptable salt of the psychedelic agent is the fumarate salt.

Parenteral routes of administration include intravenous, intramuscular, subcutaneous, intranasal, transmucosal, sublingual, rectal, and transdermal administration, and inhalation. Any parenteral routes capable of administration over a period of about 5 to about 15 minutes are suitable for use in the present invention. Preferred routes of parenteral administration according to any aspect of the present invention are selected from intravenous infusion, intramuscular infusion, subcutaneous infusion, intranasal, transmucosal, and transdermal administration, and inhalation. Intravenous infusion is a particularly preferred route of administration. Intramuscular administration is a particularly preferred route of administration. The skilled person will appreciate that the delivery device for use in the present invention is selected depending on the route of administration of the dose of a psychedelic agent.

When the parenteral route of administration is intravenous, the delivery device may comprise an infusion bag or a syringe, and may preferably further comprise a syringe pump. Where two syringe pumps are used to administer the intravenous infusion, the syringe pumps may be joined via a 3-way tap into a single cannula. When the parenteral route of administration is intramuscular the delivery device may comprise a syringe. When the parenteral route is intranasal, the delivery device may comprise a pump-action spray means. When the parenteral route is transmucosal, the delivery device may comprise an oromucosal film. When the parenteral route is transdermal, the delivery device may comprise a transdermal patch. When the route of administration is inhalation, the delivery device may comprise a metered dose inhaler, or a vaporiser, or a nebuliser.

Dosage forms suitable for parenteral administration have a pH of about 3 to 9 and, for liquid formulations, an osmolality of about 250 to about 600 mOsm/Kg. pH values above 9 are reported by I. Usach et al. in Adv. Ther., 36, 2986-2996 (2019) to relate to tissue necrosis (death of cells within the tissue), whereas values lower than 3 are reported to cause pain and phlebitis (inflammation of veins). Osmolality values greater than 600 mOsm/Kg are also reported to cause pain. Usach et al also recommend that parenteral formulations should be formulated as isotonic solutions (osmolality of about 300 mOsm/Kg), proposing an upper limit of 600 mOsm/Kg to minimise pain.

Osmolality is formally defined as the quotient of the negative natural logarithm of the rational activity of water and the molar mass of water, as represented by formula:

$$osmolality = \frac{- {lna}_{w}}{18.015} ; {a}_{w} = \frac{p}{{p}^{*}}$$

where p is the partial vapour pressure of water in the solution and p* is the partial vapour pressure of pure water. In simpler terms, osmolality is the number of osmotically active particles (the number of solute particles) in 1 kg of a solution. Thus, osmolality is a function only of the number of particles, and is not related to particle molecular weight, size, shape, or charge (see D. K. Faria et al., M. E. Mendes and N. M. Sumita, J. Bras. Patol. Med. Lab., 53, 1, 38-45 (2017) for a review of the measurement of serum osmolality). For example, one mole of a non-dissociating substance (e.g., DMT as a free base) dissolved in 1 kg of water has an osmolality of 1 Osm/kg (1000 mOsm/kg), whilst one mole of a substance that dissociates into two separate species in solution (e.g., DMT fumarate) dissolved in 1 kg of water has an osmolality of 2 Osm/kg (2000 mOsm/kg).

Where a first solution is defined herein to be isotonic with a second solution, the solutions have the same osmolality. For example, where a formulation is defined to be isotonic with human blood serum, the formulation has the same osmolality as human blood serum. Human blood serum typically has an osmolality of about 275 to about 300 mOsm/Kg (L. Hooper et al., BMJ Open, 2015; 5 (10): e008846).

Suitable formulations for injection according to the present invention are described in WO2022/043227 and U.S. Pat. No. 11,406,619. Suitable formulations for administration by inhalation in accordance with the invention are described in WO2022/117640. Suitable formulations for transdermal administration are described in US20210346347 and Ser. No. 17/866,477. Other suitable formulations according to the present invention are described in co-pending patent application U.S. Ser. No. 17/574,424. The disclosures of each of these patent applications are hereby incorporated by reference in their entireties.

In some embodiments, the parenteral formulation for use in the present invention comprises a salt of the psychedelic agent, a base agent, water and optionally a buffer which is separate to the salt, wherein the formulation has a pH of from about 5 to about 6, a concentration of about 10 mg/ml as freebase or greater, and an osmolality of from about 250 to about 350 mOsm/Kg; and wherein the formulation comprises a dose of the optionally substituted dimethyltryptamine compound within a volume of 5 ml or less.

The base agent adjusts the pH of the formulation to the required pH range, for example from pH 5 to pH 6. The pH of a formulation including the optionally substituted dimethyltryptamine salt, water, and a buffer is often low, e.g., less than pH 5, and so a pH adjustment with a base agent may be required. The skilled person is able to assess suitable base agents to adjust the pH of the solution without risk of degradation of the optionally substituted dimethyltryptamine salt. The base agent may be sodium hydroxide or potassium hydroxide.

The parenteral formulation optionally comprises a buffer, which is separate to the salt, i.e., the buffer is not merely a counterion to the psychedelic agent. For example, where the salt is N,N-dimethyltryptamine fumarate (i.e., the fumaric acid salt of N,N-dimethyltryptamine), an amount of buffer may be required over and above the buffer effect provided by the fumarate salt. The term “buffer” is well known in the art and refers to a chemical which, on inclusion within a formulation, resists a change in pH on addition of acid or base to the formulation. Within a formulation, a buffer comprises a weak acid and its conjugate base. A suitable buffer comprises an acid with a pKa value that lies within +1 of the desired pH of the formulation. For example, if the desired pH of the formulation is about 5.0, a suitable buffer comprises a weak acid with a pKa value of from about 4.0 to about 6.0. If the acid of a buffer has more than one pKa value (i.e., each molecule of the acid is able to donate more than one proton), in order for the buffer to be suitable, at least one of the pKa values lies within the desired pH range.

The weak acid and conjugate base of the buffer are in equilibrium with one another. In accordance with Le Chatelier's principle (if a constraint (such as a change in concentration of a reactant) is applied to a system in equilibrium, the equilibrium will shift so as to counteract the effect of the constraint), addition of acid or base to the formulation shifts the position of equilibrium in favour of the conjugate base or weak acid, respectively. Consequently, the concentration of free protons in the formulation (and thus the pH) is relatively unchanged.

Suitable buffer systems comprise an acetate salt and acetic acid (pKa=4.75); a citrate salt and citric acid (pKa=3.13, 4.76 and 6.40); and a phosphate salt and phosphoric acid (pKa=2.14, 7.20 and 12.37); or mixtures thereof. The pKa values cited herein are those reported at 25° C. in water. Typically, the buffer comprises only one of the pairs listed above, i.e., one acid and its conjugate base.

In some embodiments, the buffer comprises an acetate salt and acetic acid; a citrate salt and citric acid; or a phosphate salt and phosphoric acid. Sometimes, the buffer comprises an acetate salt and acetic acid; or a citrate salt and citric acid. In some embodiments, the buffer comprises an acetate salt and acetic acid, often sodium acetate and acetic acid, or potassium acetate and acetic acid.

The concentration of buffer within the formulation is typically sufficient to resist significant pH change of the formulation on storage of the formulation for two weeks (i.e., the pH typically fluctuates less than about 0.1 pH unit), The skilled person is able to assess suitable buffer concentrations and to achieve this. Often, the concentration of buffer is from about 15 mM to about 75 mM, such as about 20 mM to about 30 mM. In some embodiments, the concentration of the buffer is about 25 mM.

Sometimes, the concentration of psychedelic agent and optional buffer in the formulation gives rise to the desired osmolality. Alternatively, the desired osmolality may be achieved by inclusion of one or more tonicity agents in the formulation. Thus, in some embodiments, the formulation further comprises a tonicity agent. A tonicity agent is defined herein as a chemical that, on inclusion within a formulation, increases the osmolality of the formulation. As described above, the osmolality is the number of osmotically active particles (the number of solute particles) in 1 kg of a solution. Thus, a chemical that acts as a solute when incorporated into the formulation lies within the definition of a tonicity agent.

In some embodiments, the formulation comprises a tonicity agent. The concentration of tonicity agent depends on the concentration of other components within the formulation, such as the psychedelic agent and buffer. For example, where the formulation without tonicity agent has an osmolality of about 60 mOsm/kg, at least about 190 mOsm/kg would be provided by a tonicity agent (e.g., 95 mM of sodium chloride). M. F. Powell, T. Nguyen and L. Baloian provide a review of excipients suitable for parenteral administration (administration other than by the mouth or alimentary canal) in PDA J. Pharm. Sci. Technol., 52, 238-311 (1998). All soluble excipients listed in this review article that can be given by the intravenous route will, when added to the formulation, contribute to the osmolality and thus can be considered tonicity agents.

Suitable excipients for use in the dose of a psychedelic agent when used in accordance with the invention may be selected from the group consisting of ethanol, citric acid, trisodium citrate, benzalkonium chloride, microcrystalline cellulose, carboxymethylcellulose sodium, chlorobutanol, edetate disodium, glycerin, hydrochloric acid, methylparaben, polyethylene glycol, propylene glycol, propylparaben, saccharin sodium, sodium bicarbonate, sodium bisulphate, sodium bisulphite, sodium chloride, sodium hydroxide, sodium metabisulphite, sodium phosphate, sodium citrate, sulphuric acid, trisodium citrate, tromethamine, and mixtures thereof.

Some excipients may act as a cosolvent. Suitable solvents or cosolvents for use in the formulations of the invention may be selected from ethanol, polyethylene glycol, propylene glycol, and mixtures thereof. In some embodiments, the formulation comprises a cosolvent. In some embodiments, the formulation does not comprise a cosolvent. In particular, when the salt of the optionally substituted dimethyltryptamine compound is a fumarate, for example N,N-dimethyltryptamine fumarate or α,α-dideutero-N,N-dimethyltryptamine fumarate, the formulation does not comprise a cosolvent.

The dosage regimen, method for treating, delivery device or parenteral formulation for use of the invention may provide a number of advantages, such as a slower onset regimen (compared with administration of a bolus administration), which may improve tolerability, and may improve the therapeutic benefit in patients, by slowing the onset of the peak experience.

Tolerability may be assessed following cessation of the subjective psychedelic experience by asking the patent the question ‘Do you wish you had not gone through that experience?’ Yes responses are deemed to indicate poor tolerability, while No responses are deemed to indicate good tolerability.

In the D'Souza et al. study (D. C. D'Souza, S. A. Syed, L. T. Flynn, H. Safi-Aghdam, N. V. Cozzi and M. Ranganathan, Neuropsychopharmacology (2022) 47:1854-1862), all three healthy volunteer participants reported a tolerability score of greater than 70 (on a score of 0-100) following a 0.3 mg/kg dose of DMT, indicating a good level of tolerability. In contrast, of the 6 patients with MDD who receive a 0.3 mg/kg dose, 3 reported a tolerability score of less than 50, indicating poor tolerability (see figure S1).

These results indicate a lower tolerability for the patient group than for the healthy volunteer group, which may be due to the patient group consisting of patients with a psychiatric or neurological disorder. Garcia-Romeu and Richards (International review of Psychiatry, Vol. 30, 2018, issue 4, pp 291-316) discuss the use of serotonergic hallucinogens in clinical interventions and state ‘issues, such as recent suicidality, drug or alcohol use disorders, dissociative disorders, and past history of trauma should be taken into account depending on the therapeutic target, as these may be exacerbated by high-dose psychedelic administration or increase risk for adverse events. ‘They further state: Psychodynamic-autobiographical experiences are predominated by emotional recollections and reflections on significant previous or current life events and relationships. This can take many forms, but often involves re-emergence of past transgressions for which the patient may harbour ongoing guilt or sorrow, grief for deceased loved ones or lost relationships, anger or forgiveness regarding unresolved traumas, and insights into one's ways of being and relating throughout one's life. These types of experiences can come about at lower doses in supportive settings, . . . . Psychodynamic-autobiographical experiences may also occur during moderate- or high-dose psychedelic therapy.’ Since patients with a psychiatric disorder, such as depressive disorder, may be more likely to present with the psychodynamic-autobiographical experiences discussed by Garcia-Romeu and Richards than healthy volunteers, therapists and psychiatrists experienced in the field of psychedelic assisted psychotherapy expect patients to be more likely to undergo a more challenging psychedelic experience than healthy volunteers, and to be less likely to report the experience as tolerable. A patient's subjective experience may be evaluated by the Challenging Experience Questionnaire (CEQ), a questionnaire designed to measure challenging psychological experiences associated with a psychedelic experience.

The onset and cessation of the psychedelic experience may be assessed by the attending clinician, psychiatrist or therapist. ‘Duration of the psychedelic experience’ as used herein refers to the time from the start of the continuous parenteral administration to the cessation of the psychedelic experience. The duration of the psychedelic experience may vary between patients. The term ‘duration’ as used herein, encompasses the mean duration of the psychedelic experience.

Therapeutic benefit or efficacy may be assessed using a rating scale. For patients with a depressive disorder such as MDD or TRD, the Montgomery-Asberg Depression Rating Scale (MADRS), the Hamilton Depression Rating Scale (HAMD-17) or the Beck's Depression Inventory (BDI-II) may be used. The rating scale is used prior to administration of the psychedelic agent, to provide a baseline assessment, and again after administration of the psychedelic agent.

In some embodiments, the psychedelic agent may be administered by intramuscular administration.

In some embodiments, the psychedelic agent may be administered as a two-phase IV infusion through a venous cannula over about 6 to about 11 min. In some embodiments, the psychedelic agent may be administered as a two-phase IV infusion through a venous cannula, wherein each phase comprises about a 5-minute infusion. In some embodiments, the psychedelic agent may be administered as a single-phase IV infusion through a venous cannula over about 6 to about 11 min, preferably about 10 minutes. Where a two-phase IV infusion is used, suitably the syringe pumps are joined via a 3-way tap into a single cannula: once infusion is complete on the first pump, the 3-way tap is tuned to position to on to allow infusion to continue from the second pump.

The term ‘single-phase’ IV infusion refers to the administration of an IV infusion by one syringe pump, with the administration of the psychedelic agent from a single syringe pump. The term ‘two-phase’ IV infusion refers to the administration of an IV infusion by two syringe pumps, with the first phase comprising administration of the psychedelic agent from the first syringe pump, followed by the second phase comprising administration of the psychedelic agent from the second syringe pump. The two-phase IV infusion is essentially continuous, with a non-material break in the infusion to change administration from the first syringe pump to the second syringe pump.

### Example 1

A double-blind, randomised, placebo-controlled study of intravenous doses of DMT fumarate, was carried out in patients with major depressive disorder, MDD, (ClinicalTrials.gov Identifier: NCT04673383, Part B). The protocol comprised the steps of:

### Study Day −1

MADRS assessment by an independent assessor

### Study Day 1

Administration of either a placebo or a dose of DMT fumarate by continuous intravenous infusion over a dosing period of about 10 minutes.
Tolerability assessment following cessation of the psychedelic experience.
   Study Day 8 (±1 day), Study Day 15 (±1 day)
MADRS assessment

### Study Day 15

Administration of a dose of DMT fumarate by continuous intravenous infusion over a dosing period of about 10 minutes.
Tolerability assessment following cessation of the psychedelic experience.
   Study Day 22 (+1 day), Study Day 29 (+2 days), Day 45 (+2 days), Day 105 (+2 days)
MADRS assessment

The administration of the study drug, DMT fumarate, in NCT04673383, Part B was in accordance with the present invention. Group A received one dose of active (DMT fumarate) on Day 1 and were assessed using MADRS at Day 8 and Day 15. Group P received a placebo on Day 1, and were also assessed at Day 8 and Day 15. This comprised the blinded phase of the study. Group P subsequently received one dose of active in the open label phase of the study on Day 15 and were assessed at Day 22 and 29. Data from this group following administration of the active dose is represented as PA.

All patient participants in this study, after receiving one dose of DMT fumarate, answered NO to the question ‘Do you wish you had not gone through that experience?’, indicating good tolerability of the psychedelic agent. In Part A of the same study, healthy adults received single escalating doses of DMT fumarate. All healthy volunteers answered “no” to the same question at the highest doses tested. The study indicates that the administration of DMT fumarate in accordance with the dosage regime was equally well tolerated by both healthy volunteers and by patients.

The participant's experience was evaluated using the Challenging Experience Questionnaire (CEQ). In Part A of the study, a mean CEQ score of 0.172 was recorded for the active group, compared with a mean of 0.052 for the placebo group. In Part B of the study, a mean CEQ score of 0.350 was reported for Group PA and 0.360 for Group A, compared with a mean CEQ score of 0.120 for Group P (placebo).

These data show that the patient cohort in Part B found the experience significantly more challenging when compared to the healthy participant cohort in Part A, with mean CEQ scores of 0.350 and 0.360 compared with 0.172. It is surprising that despite the more challenging experience reported by patients, the dosage regime of the present invention was equally well tolerated by both healthy volunteers and by patients.

Furthermore, no drug related serious adverse events had been reported at the date of filing the present patent application.

In contrast, the data reported by D'Souza, as discussed above, indicate that DMT administered as an intravenous push (bolus) was not well tolerated by the patient group. Overall tolerability was assessed on a scale of 0 to 100, with 0=intolerable, and 100=well tolerated. Three patients who received a dose of 0.3 mg/kg, which is stated to have been chosen to reliably induce a psychedelic experience, reported a tolerability score under 50, indicating that the dosage regime was poorly tolerated (Figure S1). Furthermore, following administration of the 0.3 mg/kg dose, the mean score in answer to the question ‘how likely are you to use this drug?’ is stated to be 22.44 on a scale of 0 to 100, again indicating a low tolerability (Table S5). It should be noted that this sample includes 3 healthy volunteers and 6 patients.

D'Souza et al report that the mean change in HAMD-17 scores between baseline (7 subjects) and 1 day after final administration of both a 0.1 mg/kg and a 0.3 mg/kg dose (6 subjects) was −4.5 (Table S6). This improvement approximates to an improvement on the MADRS scale of −3.9.

Blinded data for all patients in the NCT04673383, Part B study show an average improvement on the MADRS score at day 8 of −7.5, an average percent reduction from baseline of −18.4%. A clinically meaningful change from baseline on the MADRS score is considered to range from a −6 to a −9 point reduction. Since these data are an average of both patients who received placebo and patients who received DMT fumarate, they indicate that efficacy was achieved following administration of DMT fumarate according to the present invention.

Unblinded data from the time points 7 and 14 days post dose (study days 8, 15, 22 and 29) are presented in TABLE 1. As described above, Group A received one dose of active (DMT fumarate) on Day 1 and were assessed at Day 8 and Day 15. Group P received a placebo on Day 1, and were also assessed at Day 8 and Day 15. Group P subsequently received one dose of active on Day 15, and were assessed at Day 22 and Day 29, with these data represented as PA.

TABLE 1

| | A | | P | | PA | |
|---|---|---|---|---|---|---|
| | Time from active dose (or placebo for group P) | | | | | |
| | 1 | 2 | 1 | 2 | 1 | 2 |
| | week | weeks | week | weeks | week | weeks |
| | study day | | | | | |
| | Day | Day | Day | Day | Day | Day |
| | 8 | 15 | 8 | 15 | 22 | 29 |
| Number of dosed subjects | 17 | 17 | 17 | 17 | 16 | 16 |
| Number of datapoints | 16 | 17 | 16 | 17 | 14 | 16 |
| Mean Baseline MADRS | 26.3 | | 25.5 | | 23.5 | |
| Mean change from baseline | −12.7 | −11.0 | −1.9 | −3.6 | −10.6 | −10.8 |
| Mean % reduction | −48.2 | −41.0 | −6.0 | −15.8 | −40.6 | −36.7 |
| % of subjects with ≥ 50% | 43.8 | 35.3 | 6.3 | 11.8 | 42.9 | 43.8 |
| reduction from BL | | | | | | |
| % of subjects with a MADRS | 43.8 | 29.4 | 12.5 | 11.8 | 42.9 | 31.3 |
| score ≤ 10 | | | | | | |

BL = baseline assessment

These data demonstrate a clear mean improvement in symptoms for subjects who received one dose of active (Groups A and PA) compared with subjects who received placebo only (Group P); with a statistically significant difference in mean change from baseline between Group A and Group P at Day 8 of −10.8 points (p=0.002); and a statistically significant difference of −7.4 points at Day 15 (p=0.02).

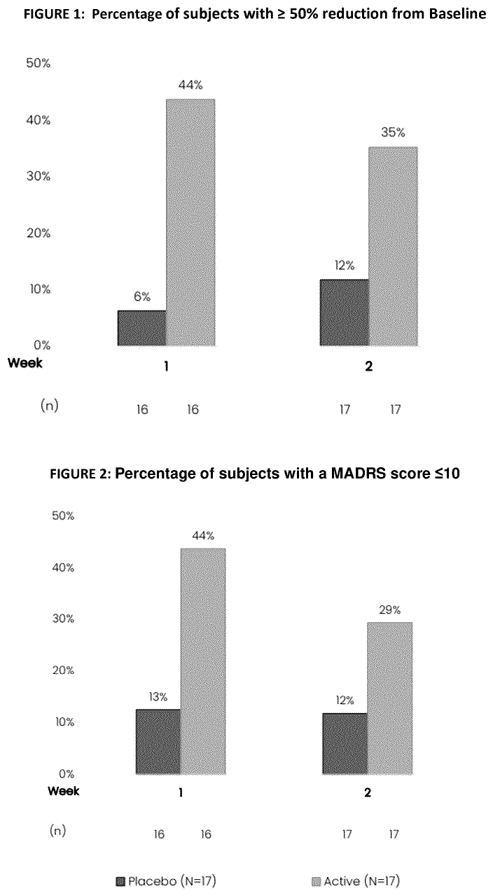

The data demonstrating the percentage of subjects from Groups A and P with ≥50% reduction from BL and the percentage of subjects Groups A and P with a MADRS score≤10 are shown in FIGS. 1 and 2, respectively.

Analysis of patient-reported depression scores corroborated the MADRS assessments conducted by independent clinical raters. Improvements in depression scores from baseline were observed across all study timepoints in patients receiving at least one dose of active, as measured by the Beck Depression Inventory (“BDI”). This includes a statistically significant improvement in depression symptoms compared to placebo at two-weeks post-dose (p=0.002). The efficacy outcomes on the BDI were consistent with MADRS, providing additional support for the rapid and sustained therapeutic profile of DMT fumarate, when administered in accordance with the invention, for the treatment of MDD.

Measures assessing patients' anxiety and wellbeing, areas which are often negatively impacted by depression, were also analysed across the study. Following one dose of DMT fumarate, administered in accordance with the invention, with supportive therapy, patients demonstrated a rapid and sustained improvement in anxiety symptoms as measured by the State-Trait Anxiety Inventory-Trait (“STAI-T”) scale. A statistically significant improvement in anxiety symptoms was observed compared to placebo at two-weeks post-dose (p=0.03). At 12-weeks following the open label dose (Group PA), a −14.2 mean change from baseline (“CFB”) was demonstrated in the patient group.

Further, a rapid and sustained improvement in wellbeing was observed following administration of at least one dose of DMT fumarate in accordance with the invention, with supportive therapy, as measured by the Warwick-Edinburgh Mental Wellbeing Scale (“WEMWBS”). The results at two-weeks following the blinded dose of DMT fumarate or placebo showed a 10.1 mean CFB in the active group compared to 0.9 in the placebo group.

Statistical analysis was conducted on the MADRS open label data (Group PA). A statistically significant difference in mean total MADRS score was observed across all open-label study timepoints (p<0.05). This analysis provides further support that a single dose of DMT fumarate, administered in accordance with the invention, is sufficient to elicit a rapid and durable antidepressant effect.

| | Blinded Phase | | Open Label Phase |
|---|---|---|---|
| | (Day 15) | | (Day 105*) |
| | Placebo | Active | PA |
| Self-Reported Depression | −3.8 | −17.4 | −19.7 |
| (BDI CFB) | | | |
| p value (BDI CFB | p = 0.002 | | n/a |
| active vs. placebo) | | | |
| Anxiety (STAI-T CFB) | −3.4 | −11.0 | −14.2 |
| p value (STAI-T CFB | p = 0.03 | | n/a |
| active vs. placebo) | | | |
| Wellbeing | 0.9 | 10.1 | 9.8 |
| (WEMWBS CFB) | | | |

### Example 2

The safety and tolerability of a single intravenous dose of a deuterated analogue of DMT is being investigated in healthy participants in an on-going clinical trial. The administration of the deuterated analogue of DMT is in accordance with the present invention. Tolerability is assessed post-psychedelic experience by the question ‘Do you wish you had not gone through that experience?’.

A 9 mg dose of the deuterated analogue was administered in accordance with the present invention to a cohort of 6 psychedelic-experienced healthy participants. Preliminary data were compared with pharmacokinetic data from Part A of NCT04673383, Cohort 1 (9 mg DMT fumarate). Comparable pharmaceutical formulations were used for both trials.

A significant increase of 4.7 fold the half-life and 1.5 fold the Intensity Rating Visual Analogue Scale (IRVAS) score was observed following administration of 9 mg of the deuterated analogue when compared with administration of 9 mg of DMT. IRVAS is a rating scale which measures the intensity of the psychedelic experience. Additionally, the perceived duration of the psychedelic experience was in the range 37-57 minutes for the deuterated analogue, compared with approximately 25 minutes for DMT. While the duration and the intensity of the psychedelic experience were shown to be significantly higher for the deuterated analogue, no difference was observed in tolerability to the psychedelic experience when compared with DMT.

These data indicate that the deuterated analogue of DMT would be equally well tolerated by a patient group.

1-25. (canceled)

26. A method for treating a psychiatric or neurological disorder in a patient, comprising
parenterally administering to the patient a dose of a psychedelic agent for a dosing period of from about 5 to about 15 minutes, wherein the psychedelic agent is selected from N,N-dimethyltryptamine, 5-methoxy-N,N-dimethyltryptamine, 4-acetoxy-N,N-dimethyltryptamine, psilocybin, psilocin, deuterated analogues thereof, and pharmaceutically acceptable salts thereof, and
wherein the parenteral administration provides a slow onset breakthrough psychedelic experience in the patient.

27. The method for treating according to claim 26, wherein the parenteral administration is selected from the group consisting of intravenous, intramuscular, subcutaneous, intranasal, transmucosal, sublingual, rectal, and transdermal administration and inhalation.

28. The method for treating according to claim 26, wherein the administration is by intravenous infusion, preferably wherein the intravenous administration is conducted using a syringe pump.

29. The method for treating according to claim 26, wherein the dosing period is from about 8 to about 12 minutes.

30. The method for treating according to claim 26, wherein the dosing period is from about 9 to about 11 minutes, or about 10 minutes.

31. The method for treating according to claim 26, comprising the parenteral administration of a total dose of the psychedelic agent selected from the group consisting of:
about 20 to about 70 mg of N,N-dimethyltryptamine, a deuterated analogue, or a pharmaceutically acceptable salt thereof;
about 15 to about 30 mg of N,N-dimethyltryptamine, a deuterated analogue, or a pharmaceutically acceptable salt thereof;
about 5 to about 15 mg of N,N-dimethyltryptamine, a deuterated analogue, or a pharmaceutically acceptable salt thereof;
about 10 to about 30 mg of 5-methoxy-N,N-dimethyltryptamine, a deuterated analogue, or a pharmaceutically acceptable salt thereof
about 1 to about 5 mg of psilocybin, a deuterated analogue, or a pharmaceutically acceptable salt thereof;
about 3 to about 15 mg of 4-acetoxy-N,N-dimethyltryptamine, a deuterated analogue, or a pharmaceutically acceptable salt thereof; and
about 1 to about 5 mg, or about 3 to about 25 mg of psilocin, a deuterated analogue, or a pharmaceutically acceptable salt thereof.

32. The method for treating according to claim 26, comprising the parenteral administration of a total dose of about 20 to about 29 mg of N,N-dimethyltryptamine, a deuterated analogue, or a pharmaceutically acceptable salt thereof.

33. The method for treating according to claim 26, comprising the parenteral administration of a total dose of about 20 to about 23 mg, or about 26 to about 29 mg of N,N-dimethyltryptamine, a deuterated analogue, or a pharmaceutically acceptable salt thereof.

34. The method for treating according to claim 26, comprising the parenteral administration of a total dose of about 7 to about 9 mg, or about 10 to about 20 mg of N,N-dimethyltryptamine, a deuterated analogue, or a pharmaceutically acceptable salt thereof.

35. The method for treating according to claim 26, comprising the parenteral administration of a total dose of about 1.5 to about 3 mg of psilocybin, a deuterated analogue, or a pharmaceutically acceptable salt thereof, or about 1.5 to about 3 mg of psilocin, a deuterated analogue, or a pharmaceutically acceptable salt thereof.

36. The method for treating according to claim 26, wherein the psychedelic agent is a compound of formula I, or a pharmaceutically acceptable salt thereof: 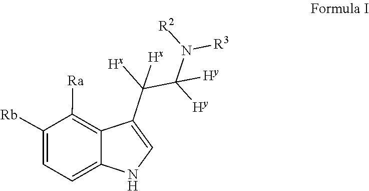
Ra is selected from H, D, —OH, —OAc and —PO₃OH, and Rb is H or D; or
Ra is H or D, and Rb is selected from H, D, and —OMe;
R² and R³ are each independently selected from C(H^{z})₃; and
each H^{x}, H^{y} and H^{z} is independently selected from protium and deuterium.

37. The method for treating according to claim 36, wherein R² and R³ are each independently selected from C(H)₃ and C(D)₃.

38. The method for treating according to claim 36, wherein R² and R³ are both C(H)₃, or R² and R³ are both C(D)₃, or R² is C(H)₃ and R³ is C(D)₃.

39. The method for treating according to claim 36, wherein each H^{x} is H, or each H^{x} is D, or one H^{x} is H and one H^{x} is D.

40. The method for treating according to claim 36, wherein each H^{y} is H, or each H^{y} is D, or one H^{y} is H and one H^{y} is D.

41. The method for treating according to claim 36, wherein each H^{x}, H^{y} and H^{z} is deuterium.

42. The method for treating according to claim 26, wherein the psychedelic agent is selected from the group consisting of: 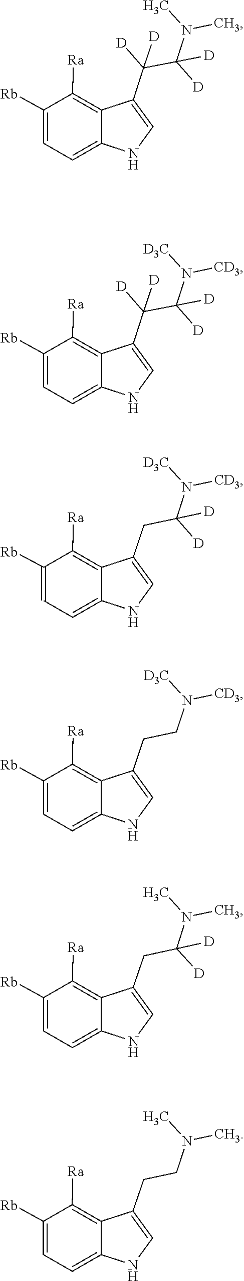
or a pharmaceutically acceptable salt thereof; wherein
Ra is selected from H, D, —OH, —OAc and —PO₃OH, and Rb is H or D; or
Ra is H or D, and Rb is selected from H, D, and —OMe.

43. The method for treating according to claim 36, wherein Ra is H.

44. The method for treating according to claim 36, wherein Rb is H.

45. The method for treating according to claim 26, wherein the parenteral administration comprises intravenous infusion by one or two syringe pumps.

46. The method for treating according to claim 26, wherein the psychiatric or neurological disorder is selected from the group consisting of (i) an obsessive compulsive disorder, (ii) a depressive disorder, (iii) an anxiety disorder, (iv) substance abuse and gambling disorders and (v) an avolition disorder, or is selected from the group consisting of major depressive disorder, treatment resistant major depressive disorder, post-partum depression, an obsessive compulsive disorder and an eating disorder such as a compulsive eating disorder.

47. The method for treating according to claim 26, wherein the psychiatric or neurological disorder is selected from the group consisting of a depressive disorder and an anxiety disorder.

48. The method for treating according to claim 26, wherein, the duration of the psychedelic experience is:
From about 15 to about 30 minutes, or from about 30 to about 90 minutes when the psychedelic agent is N,N-dimethyltryptamine, a deuterated analogue, or a pharmaceutically acceptable salt thereof; or
From about 15 to about 45 minutes, or about 45 minutes to about 180 minutes when the psychedelic agent is selected from psilocybin, 4-acetoxy-N,N-dimethyltryptamine, 5-methoxy-N,N-dimethyltryptamine, psilocin, a deuterated analogue thereof, or a pharmaceutically acceptable salt thereof; or
From about 20 to about 30 minutes wherein the psychedelic agent is N,N-dimethyltryptamine or a pharmaceutically acceptable salt thereof; or
From about 60 to about 90 minutes wherein the psychedelic agent is a deuterated analogue of N,N-dimethyltryptamine, or a pharmaceutically acceptable salt thereof.

49. The method for treating according to claim 26, wherein the psychedelic agent is N,N-dimethyltryptamine, or a pharmaceutically acceptable salt thereof, and the duration of the psychedelic experience is about 20 to about 30 minutes; or the psychedelic agent is a deuterated analogue of N,N-dimethyltryptamine, or a pharmaceutically acceptable salt thereof, and the duration of the psychedelic experience is about 60 to about 90 minutes.

50. The method for treating according to claim 26, where the treatment of the psychiatric or neurological disorder comprises psychedelic assisted psychotherapy.
